(19) United States
(12) Patent Application Publication
(10) Pub. No.: US 2014/0080154 A1
(43) Pub. Date: Mar. 20, 2014

(54) IMMUNODIAGNOSTIC METHOD FOR DIAGNOSING AUTO-IMMUNE SYSTEMIC SCLEROSIS (SSC) AND SYSTEMIC LUPUS ERYTHEMATOSUS (SLE)

(71) Applicants: Francesco Curcio, Udine (UD) (IT); Giacomo Cattaruzzi, Udine (IT); Massimo Moretti, Udine (IT); Francesco Vitrani, Udine (IT); Raffaella Scorza, Milano (IT)

(72) Inventors: Francesco Curcio, Udine (UD) (IT); Giacomo Cattaruzzi, Udine (IT); Massimo Moretti, Udine (IT); Francesco Vitrani, Udine (IT); Raffaella Scorza, Milano (IT)

(73) Assignee: VIVABIOCELL S.P.A., Udine (UD) (IT)

(21) Appl. No.: 14/007,044

(22) PCT Filed: Apr. 13, 2012
(86) PCT No.: PCT/EP12/56800

§ 371 Date: Dec. 5, 2013

Related U.S. Application Data
(60) Provisional application No. 61/475,907, filed on Apr. 15, 2011.

(51) Int. Cl. G01N 33/569

(52) U.S. Cl. CPC G01N 33/56966
USPC 435/7.92

(57) ABSTRACT

The invention relates to a method for the identification of patients affected by Systemic Sclerosis (SSc) (early, limited cutaneous, diffuse forms) or Systemic Lupus Erythematosus (SLE). Among SSc and SLE patients, this method allows identification of presence of vascular ulcerations in SSc, and Raynaud's phenomenon in SLE, respectively.

### FIELD OF THE INVENTION

The present invention relates to a method for the identification of patients affected by Systemic Sclerosis (SSc) (early, limited cutaneous, diffuse forms) or Systemic Lupus Erythematosus (SLE). Among SSc and SLE patients, this method allows identification of presence of vascular ulcerations in SSc, and Raynaud's phenomenon in SLE, respectively.

### BACKGROUND OF THE INVENTION

Systemic sclerosis (SSc) is a chronic multisystem disorder of unknown etiology characterized clinically by thickening of the skin caused by accumulation of connective tissue and by structural and functional abnormalities of visceral organs, including gastrointestinal tract, lungs, heart, and kidneys. Vascular damage, immune activation, and excessive synthesis and deposition of extracellular matrix are prominent features of SSc.

SSc has a worldwide distribution and affects all races. Overall women are affected approximately three times as often as men and even more often during the mid to late reproductive years (≧8:1). The annual incidence has been estimated to be 19 cases per million individuals. The prevalence reported for SSc is between 50 and 300 per million individuals (1).

The outstanding feature of SSc is overproduction and accumulation of collagen and other extracellular matrix proteins, including fibronectin, tenascin, fibrillin-1, and glycosaminoglycans, in skin and other organs. The disease process involves immunologic mechanisms, vascular endothelial cell activation and/or injury, and activation of fibroblasts resulting in production of excessive collagen.

Presence of serum autoantibodies has been established as strong predictor of disease outcome and the pattern of organ complications in patients; however, the pathogenic role of autoantibodies in scleroderma remains unclear, although there is a growing body of evidence that they are not just markers of disease, but also have a role in pathogenesis. Improved methods to detect and evaluate autoantibodies offer a real opportunity for risk stratification in sclerodermic patients, most of whom can be defined by their serological profile at initial presentation (2).

The three types of anti-nuclear antibodies (ANA) that are most frequently associated with SSc—anti-centromere antibodies (ACA), anti-topoisomerase antibodies (ATA) and RNA-polymerase III antibodies (ARA)—are found in over 50% of patients with the disease. They are highly specific and are generally present exclusively of each other, although a small proportion of SSc patients can be positive for both ACA and ATA. Even though multiply cohorts study have focused on the association of specific autoantibodies with clinical presentation in SSc patients, the use of different testing techniques and variable organ complication definitions make the interpretation of these studies difficult. The sensitivity and specificity of different antibodies in diagnosing SSc and predicting certain clinical features are affected considerably by the methods used for detection (indirect immunofluorescence, immunoblotting, ELISA, immunoprecipitation).

Frequencies of the different autoantibody types in SSc patients and their main clinical associations are outlined in Table 1.

TABLE 1
(Adapted from ref. 2). Frequency of auto-antibodies and their
main clinical associations within Scleroderma.

| | Frequency | Clinical associations | |
|---|---|---|---|
| Auto-antibody | (%) in SSc | (SSc) | Reference |
| Anti-centromere | 18-39 | lcSSc, PF, PBC | (6) |
| Anti-Scl70 | 9-39 | dSSc > lcSSc, PF | (7) |
| Anti-RNApol | 4-25 | dSSc, SRC | (8) |
| Anti-Th/To | 1-7 | lcSSc, PF (+Sjögren, | (9) |
| | | RA . . . ) | |
| Anti-U3RNP | 1-6 | dSSc > lcSSc, severe | (10) |
| Anti-PM-Scl | 1-6 | PF, arthritis, polymiositis | (11) |
| Anti-Ku | 1-3 | Muscle and joint | (12) |
| | | involvement | |
| Anti-U1RNP | 5-35 | Overlap syndromes | (13) |
| Anti-U11/U12RNP | 1-5 | PF | (14) |

Scl70: Topoisomerase I,
RNApol: RNA polymerase,
Th/To: small nuclear ribonucleoprotein components of RNAse MRP and RNAse P,
U3RNP: fibrillarin,
PM-Scl: human exosome proteins Scl-75 and Scl-100,
U1/11/12RNP: components of spliceosome.
Abbreviations:
lcSSc, limited cutaneous SSc;
dSSc, diffuse SSc;
PF, pulmonary fibrosis;
PBC, primary biliary cirrhosis;
SRC, scleroderma renal crisis;
RA, rheumatoid arthritis.

Systemic sclerosis is also characterized by injury to vascular wall and extensive damage of the microvessels. The injury of the vascular wall is characterized by the formation of megacapillaries and avascular areas. The reduced capillary density leads to clinical manifestations such as digital ulcers. Despite of reduced blood flow and reduced partial oxygen pressure levels, paradoxically there is no evidence for a sufficient angiogenesis in the skin of patients with SSc. Angiogenesis is strongly disturbed in SSc, as demonstrated by Nailfold Video-Capillaroscopy changes, vessel damages evolve progressively from early to late stages and is characterized by different morphological characteristics. Almost all patients develop Raynaud's phenomenon, which, together with structural vasculopathy, results in ulceration and critical digital ischemia. Many of the severe internal organ complications of SSc are vascular, including pulmonary arterial hypertension (PAH) and scleroderma renal crisis. Structural vascular damage occurs in many vascular beds and contribute to pulmonary, renal, cardiac and gastrointestinal complications (3).

Systemic lupus erythematosus (SLE) is a prototypic autoimmune disease characterized by the production of antibodies to components of the cell nucleus in association with a variety of clinical manifestations. The primary pathological findings in patients with SLE are those of inflammation, vasculitis, immune complex deposition, and vasculopathy. The exact etiology of SLE is unknown (4).

Ninety percent of patients are fertile women; people of both genders, all ages, and all ethnic groups are susceptible. Prevalence of SLE in the United States is 15 to 50 per 100,000; the highest prevalence among ethnic groups is in African Americans.

SLE is caused by interactions between susceptibility genes and environmental factors, resulting in abnormal immune responses. The immune responses include hyperactivity and hypersensitivity of T and B lymphocytes and ineffective regulation of antigen availability and of ongoing antibody responses. The end result of these abnormalities is a sustained production of pathogenic autoantibodies and formation of immune complexes that bind target tissues, resulting in sequestration and destruction of Ig-coated circulating cells, fixation and cleaving of complement proteins, and release of chemotaxins, vasoactive peptides, and destructive enzymes into tissues. In patients with SLE many autoantibodies are directed against DNA/protein or RNA/protein complexes such as nucleosomes, some nucleolar RNA, and spliceosomal RNA (5).

Antinuclear antibodies (ANA) are positive in 95% of patients during the course of disease. High titer of IgG antibodies to double-stranded DNA and antibodies to the Sm antigen are both specific for SLE and, therefore, favor the diagnosis in the presence of compatible clinical manifestations. The presence in an individual of multiple autoantibodies without clinical symptoms should not be considered diagnostic for SLE, although such persons are at increased risk.

The present invention provides a cell-extract based method which is capable of identify SSc and SLE patients with very high specificity.

### DESCRIPTION OF THE INVENTION

It is an object of the instant invention an in vitro method for the diagnosis of Systemic Sclerosis (SSc) and/or Systemic Lupus Erythematosus (SLE) in a subject comprising the step of:
reacting a test biological fluid sample taken from said subject with a mammalian cell nuclear extract reagent previously immobilized onto a solid support, under condition allowing an antigen-antibody binding;
reacting a reference sample with a further mammalian cell extract reagent previously immobilized onto a solid support, under condition allowing an antigen-antibody binding;
washing;
adding detecting means able to detect the antigen-antibody binding, if occurred, and generating detecting values;
comparing detecting values of the test biological fluid sample with detecting values of the reference serum sample to obtain a mean fold reactivity value;
checking if the mean fold reactivity value is higher or lower of a predefined cut-off value.

The method of the invention may be performed on any test biological fluid sample as but not limited to blood, plasma serum, optionally stored, frozen or lyophilized.

The mammalian cell nuclear extract reagent may be obtained by any mammalian cell, either stabilized cell lines or primary cells. Examples of suitable cell lines are CHO, HeLa, 293 and MRC-5.

According to the invention the cell nuclear extract reagent is obtained by:

a) harvesting mammalian cells,

b) suspending harvested mammalian cells in a solution able to lyse cells maintaining intact nuclei, to get a mammalian cell lysate with intact nuclei;

c) pelleting the mammalian cell lysate and washing pelleted intact nuclei,

d) lysing nuclei in a proper solution to get a mammalian nuclear lysate, and

e) sonicating and centrifuging the nuclear lysate, discarding insoluble matter.

Preferably the solution in b) is a buffered, low salt hypotonic solution, completed with a protease inhibitors mix.

Preferably the solution in d) is a buffered, high salt hypertonic solution, completed with a protease inhibitors mix.

Preferably detecting means are peroxidase conjugated secondary antibodies, more preferably peroxidase-conjugated anti-human IgGs.

According to the invention the cut-off value is approximately 1.35 fold with respect to reference.

The method is particularly useful for identifying the presence of vascular ulceration in SSc and Raynaud's phenomenon in SLE. SSc belongs to the group of: early SSc, limited cutaneous SSc, diffuse SSc, CREST.

It is a further object of the invention a kit for the diagnosis of Systemic Sclerosis (SSc) and/or Systemic Lupus Erythematosus (SLE) univocally addressed to the exploitation of the method as above disclosed.

In the present invention a cell nuclear extract reagent is immobilized onto a solid support, i.e. an ELISA plate, and is directly probed by serum samples.

Thus, the method detects the presence of (auto)antibodies bound to the immobilized cell nuclear extract.

The execution of this method does not require specific technical expertise, and a typical ELISA technical equipment would be sufficient to obtain affordable results.

For the execution of this method, the cell nuclear extract may be obtained by standard cultured mammalian cell lines, as for not limiting examples, CHO, Hela, 293, MRC-5 cell lines. It is worth to note that said cell lines are from different mammalian and tissue origins.

Human sera samples are required. It is not necessary to expressly obtain fresh material, since sample freezing does not influence performance. Samples are advantageously diluted in Dilution Buffer.

The procedure is easy, fast and reliable, and could be summarized with the following steps:
Equilibrate plate in dilution buffer;
Prepare three different serum sample dilutions (1:100, 1:250 and 1:500) in dilution buffer;
Prepare three different reference serum dilutions (1:100, 1:250 and 1:500) in dilution buffer;
Add samples to plate and incubate 1 h at room temperature;
Wash three times in Washing buffer;
Add peroxidase-conjugated anti-human IgG (or other secondary antibody, depending on detection method) and incubate 1 h at room temperature;
Wash three times in Washing buffer;
Add substrate and read appropriate absorbance.

### DETAILED DESCRIPTION OF THE INVENTION

The invention will be now described by non limiting examples referring to the following figures:

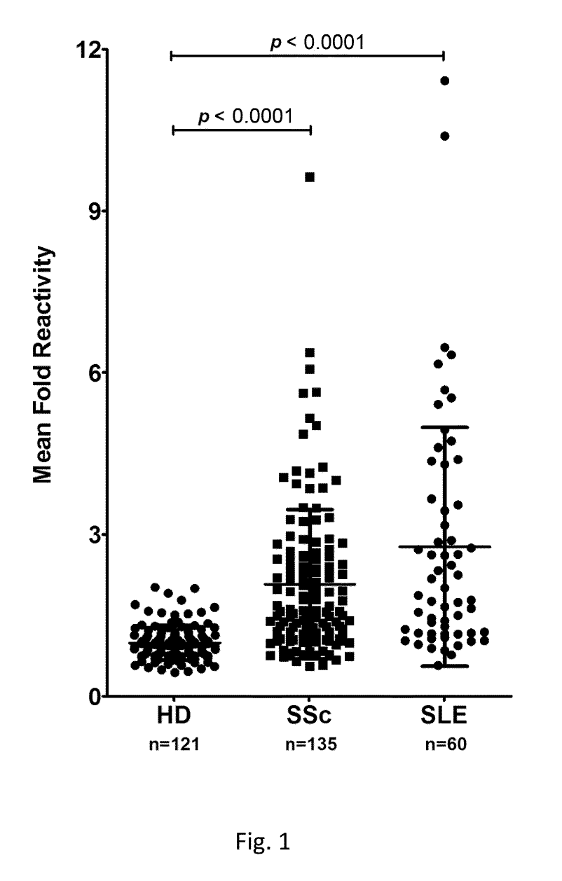

FIG. 1. Plot distribution of Mean Fold Reactivity results for n=121 Healthy Donors, n=135 SSc patients and n=60 SLE patients with respect to the reference serum. Mean levels and standard deviations are shown as lines. One-tailed Student's t test result indicates high significant difference between the two populations.

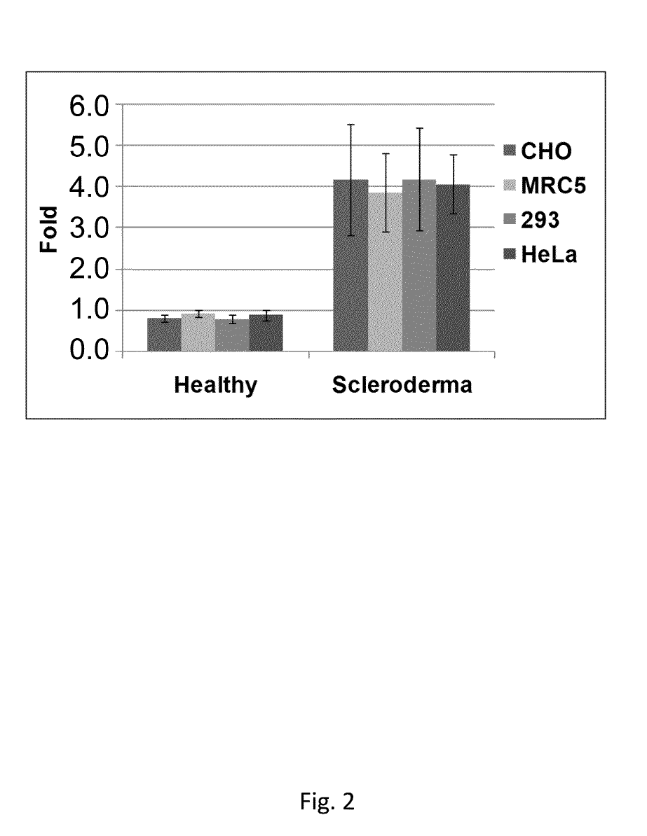

FIG. 2: Systemic Autoimmune Angiopathy Test (SAAT) test results of one representative healthy blood donor subject and one representative scleroderma donor subject. Extracts from four different cell lines (CHO, MRC-5, 293 and HeLa) were used as a substrate for preparing plates and performing assay. Fold increase in reactivity with respect to a Reference Mix (corresponding to a mix of one hundred different sera from different healthy blood donors) is indicated. Fold increase of the representative scleroderma donor subject is comparable for all of the different cell lines (no significant differences are found).

### MATERIAL AND METHODS

### Cells

Cells tested for the obtaining of extracts suitable for the execution of the SAAT test include: cell line CHO (CHO-K1, ATCC no: CCL-61, from Cricetulus griseus ovary), cell line HeLa (HeLa, ATCC no: CCL-2, from Homo sapiens cervix adenocarcinoma), cell line 293 (293 or HEK-293, ATCC no: CRL-1573, from Homo sapiens embryonic kidney) and cell line MRC-5 (MRC-5, ATCC no: CCL-171 from Homo sapiens lung fibroblasts). Extracts from these cells were comparable in terms of performance of the SAAT test (see FIG. 2).

### Preparation of Nuclear Extracts

Cells are harvested and washed twice with PBS (137 mM Sodium Chloride, 8.1 mM Dibasic Sodium Phosphate, 2.7 mM Potassium Chloride, 1.76 mM Monobasic Potassium Phosphate, final pH=7.4), then resuspended in 50 microliters per million cells of ice cold “Solution 1” (10 mM 4-(2-hydroxyethyl)-1-piperazineethanesulfonic acid (HEPES) pH=7.9, 10 mM Potassium Chloride, 0.1 mM Magnesium Chloride, 0.1 mM Ethylenediaminetetraacetic acid (EDTA) pH=8.0, 0.1 mM Dithiothreitol, 100 mcM 4-(2-Aminoethyl) benzenesulfonyl fluoride hydrochloride (AEBSF), 80 nM Aprotinin, 2 mcM Leupeptin, 4 mcM Bestatin, 1.5 mcM Pepstatin A, 1.4 mcM trans-Epoxysuccinyl-L-leucylamido(4-guanidino)butane (E-64)), until suspension appears homogeneous. Suspension is then centrifuged at 800×g for 10 minutes in a controlled temperature centrifuge, at 4° C. The resulting supernatant corresponds to cytosolic extract, while pellet corresponds to intact nuclei. The supernatant is discarded, and the pellet is gently resuspended in the same volume of ice cold Solution 1 than in the previous step. Suspension is then centrifuged at 800×g for 10 minutes in a controlled temperature centrifuge, at 4° C. The supernatant is discarded, then the pellet is resuspended again in the same volume of ice cold Solution 1, then centrifuged again in the same conditions as indicated above. The pellet is then resuspended in the same volume of ice cold “Solution 2” (5% volume/volume Glycerol, 20 mM 4-(2-hydroxyethyl)-1-piperazineethanesulfonic acid (HEPES) pH=7.9, 420 mM Sodium Chloride, 1.5 mM Magnesium Chloride, 0.1 mM Ethylenediaminetetraacetic acid (EDTA) pH=8.0, 0.1 mM Dithiothreitol, 100 mcM 4-(2-Aminoethyl) benzenesulfonyl fluoride hydrochloride (AEBSF), 80 nM Aprotinin, 2 mcM Leupeptin, 4 mcM Bestatin, 1.5 mcM Pepstatin A, 1.4 mcM trans-Epoxysuccinyl-L-leucylamido(4-guanidino)butane (E-64)), until suspension appears homogeneous. Suspension is left 30 minutes on ice; during this time, suspension is vortexed, for 10 seconds at 2000 revolutions per minute, every 5 minutes for a total of six times. Suspension is then sonicated for 30 seconds under mild sonication conditions (constant duty cycle, 15% output power), then left on ice for 30 seconds; this sonication cycle is repeated for a total of eight times. Suspension is then centrifuged at 20.000×g for 30 minutes in a controlled temperature centrifuge, at 4° C. The resulting supernatant corresponds to nuclear extracts, while pellet corresponds to membranes and insoluble matter. The supernatant is saved, then centrifuged at 20.000×g for 10 minutes in a controlled temperature centrifuge, at 4° C. Finally, the supernatant is saved and stored at −80° C. or below.

### Immobilization of the Nuclear Extract into ELISA Plate

Polystyrene, “high binding” multiwell plate is washed once with PBS, for 5 minutes onto a vibrating orbital platform shaker at 300 revolutions per minute at room temperature (20° C.-25° C.). Plate is coated with a total of 2 micrograms extracts per well, dissolved in PBS in a final volume of 50 microliters per well; plate is left 60 minutes onto a vibrating orbital platform shaker at 150 revolutions per minute in order to allow binding of extracts to wells. Plate is then washed once with PBS, for 5 minutes onto a vibrating orbital platform shaker at 300 revolutions per minute at room temperature, then incubated with 300 microliters per well of a 1% Bovine Serum Albumin solution in PBS, for 60 minutes onto a vibrating orbital platform shaker at 150 revolutions per minute in order to saturate reactive sites. Plate is finally washed once with PBS, for 5 minutes onto a vibrating orbital platform shaker at 300 revolutions per minute at room temperature.

### Immunoassay

Sera from blood donors are diluted in 1% Bovine Serum Albumin solution in PBS, and incubated for 60 minutes onto a vibrating orbital platform shaker at 150 revolutions per minute. Then, plate is washed three times with 200 microlitres of a 0.05% Polyoxyethylene (20) sorbitan monolaurate (Tween 20) solution in PBS per well, for 5 minutes onto a vibrating orbital platform shaker at 300 revolutions per minute at room temperature per wash. Then, plate is incubated with 200 microliters of a 4 mg/ml peroxidase-conjugated anti-human immunoglobulin G solution, dissolved in 1% Bovine Serum Albumin solution in PBS per well, for 60 minutes onto a vibrating orbital platform shaker at 150 revolutions per minute. Subsequently, plate is washed three times with 200 microlitres of a 0.05% Polyoxyethylene (20) sorbitan monolaurate (Tween 20) solution in PBS per well, for 5 minutes onto a vibrating orbital platform shaker at 300 revolutions per minute at room temperature per wash. Plate is then incubated with 100 microliters 3,3′,5,5′-Tetramethylbenzidine (TMB) per well, for 5 minutes onto a vibrating orbital platform shaker at 150 revolutions per minute at room temperature. Finally, 100 microliters of a 500 mM Sulphuric Acid solution are dispensed into each well, in order to block the color development. Absorbance at 450 nanometers is measured for each well.

### Results

The method provides qualitative results in terms of distinguishing healthy individuals and SSc/SLE subjects; it is quantitative in terms of overall reactivity, that is, amount of (auto)antibodies bound to immobilized phase, but not in terms of amount of serum (auto)antibodies.

This method overcomes the inter-assay variations by testing the Reference healthy mixture in each experiment.

The numeric result obtained indicates the “mean fold reactivity” of a subject's serum with respect to the Reference serum, which equals to 1. It is calculated by:
dividing each 1:100, 1:250, 1:500 diluted sample absorbance value by 1:100, 1:250, 1:500 diluted Reference absorbance value, respectively. This gives three “fold reactivity” values. Note that Reference would result equal to 1.
calculating mean of the three values obtained. This indicates “mean fold reactivity”.

Intra-assay variations have been demonstrated to be not significant.

The numeric results of a study involving 121 healthy individuals, 135 SSc patients and 60 SLE patients indicated that this method was capable to significantly distinguish the two patients populations with respect to the healthy individuals one (FIG. 1).

A total number of 135 serum samples from clinically characterized scleroderma patients (early SSc, limited cutaneous SSc, diffuse SSc, CREST), 60 serum samples from clinically characterized systemic lupus erithematosus patients and 121 serum samples from healthy donors were tested for fold-reactivity with respect to a Reference healthy mixture.

Sensibility and specificity for positivity to this test have been calculated via ROC analysis at the cut-off value of 1.35 fold with respect to the Reference (Table 2).

TABLE 2
Sensitivity and specificity values for positivity
of SSc and SLE patients to this test.

| | Sensitivity | Specificity |
|---|---|---|
| SSc | 63.7% | 90.4% |
| SLE | 68.4% | 90.4% |

Following ROC statistical analysis, a “mean fold reactivity” value of 1.35 should be considered the cut-off for positivity to this test. However, results ranging from 1.28 to 1.42 (a 5% deviation from 1.35) are to be considered as “border-line” and, in those cases, it is suggested to repeat the test and/or to perform other tests in order to assess presence of serum autoantibodies.

This method is also capable of identify, within SSc patients (limited cutaneous and diffuse forms), subjects showing vascular ulcerations also at early stages; moreover, positivity to this test correlates with presence of Raynaud's phenomenon in SLE subjects (Table 3). A total number of 81 serum samples from scleroderma patients (limited cutaneous and diffuse forms) clinically characterized for vascular ulcerations presence and 50 SLE patients clinically characterized for Raynaud phenomenon presence were tested for fold-reactivity with respect to a Reference healthy mixture.

TABLE 3
Percentage of occurrence in 57 limited cutaneous scleroderma
(lcSSc) patients, 24 diffuse scleroderma (dSSc) patients and
50 Systemic Lupus Erythematosus (SLE) patients of positivity
or negativity to this test (Test+ or Test− respectively)
with respect to presence or absence of vascular ulcerations
phenomena (Ulc+ or Ulc− respectively) in SSc, and
with respect to presence or absence of Raynaud's
phenomenon (Ray+ or Ray− respectively) in SLE.

| | |
|---|---|
| lcSSc (n = 57) | dSSc (n = 24) |

| | Test− | Test+ | Test− | Test+ |
|---|---|---|---|---|
| Ulc− | 19.3% | 26.3% | 16.7% | 8.3% |
| Ulc+ | 12.3% | 42.1% | 20.8% | 54.2% |

SLE (n = 50)

| | Test− | Test+ |
|---|---|---|
| Ray− | 30.0% | 36.0% |
| Ray+ | 8.0% | 26.0% |

Sensibility and specificity for correlation of positivity to this test with presence of vascular complications (ulcerations) in SSc, and presence of Raynaud's phenomenon in SLE, have been calculated (Table 4).

TABLE 4
Sensitivity and specificity values for prediction of vascular
complications in SSc and SLE patients with this test.

| | Sensitivity | Specificity |
|---|---|---|
| lcSSc | 77.4% | 42.3% |
| dSSc | 72.2% | 66.6% |
| SLE | 76.4% | 45.4% |

The specificity of the identification of vascular complications phenomena of autoimmune origin is also verified by the testing a total of n=36 subjects affected by cryoglobulinemia with vascular ulcerations to the upper and lower limbs (n=13) and idiopathic venous thromboembolism with the presence of anti-endothelium antibodies (n=23). Overall, 88.9% of these subjects results negative to the SAAT test.

### REFERENCES

(1) Chifflot H, Fautrel B, Sordet C, Chatelus E, Sibilia J. Incidence and prevalence of systemic sclerosis: a systematic literature review. Semin Arthritis Rheum. 2008 February; 37(4):223-35.

(2) Nihtyanova S I, Denton C P. Autoantibodies as predictive tools in systemic sclerosis. Nat Rev Rheumatol. 2010 February; 6(2):112-6.

(3) Guiducci S, Giacomelli R, Cerinic M M. Vascular complications of scleroderma. Autoimmun Rev. 2007 September; 6(8):520-3. Epub 2007 Jan. 12.

(4) Stacy P Ardoin and David S Pisetsky. Developments in the scientific understanding of lupus. Arthritis Research & Therapy 2008, 10:218.

(5) Davidson A, Diamond B: Autoimmune diasease. N Engl J Med. 2001, 345:340.

(6) Koenig M, Joyal F, Fritzler M J, Roussin A, Abrahamowicz M, Boire G, Goulet J R, Rich E, Grodzicky T, Raymond Y, Senécal J L. Autoantibodies and microvascular damage are independent predictive factors for the progression of Raynaud's phenomenon to systemic sclerosis: a twenty-year prospective study of 586 patients, with validation of proposed criteria for early systemic sclerosis. Arthritis Rheum. 2008 December; 58(12):3902-12.

(7) Basu D, Reveille J D. Anti-scl-70. Autoimmunity. 2005 February; 38(1):65-72.

(8) Kuwana M, Okano Y, Pandey J P, Silver R M, Fertig N, Medsger T A Jr. Enzyme-linked immunosorbent assay for detection of anti-RNA polymerase III antibody: analytical accuracy and clinical associations in systemic sclerosis. Arthritis Rheum. 2005 August; 52(8):2425-32.

(9) Mitri G M, Lucas M, Fertig N, Steen V D, Medsger T A Jr. A comparison between anti-Th/To- and anticentromere antibody-positive systemic sclerosis patients with limited cutaneous involvement. Arthritis Rheum. 2003 January; 48(1):203-9.

(10) Tormey V J, Bunn C C, Denton C P, Black C M. Anti-fibrillarin antibodies in systemic sclerosis. Rheumatology (Oxford). 2001 October; 40(10):1157-62.

(11) Hanke K, Brückner C S, Dähnrich C, Huscher D, Komorowski L, Meyer W, Janssen A, Backhaus M, Becker M, Kill A, Egerer K, Burmester G R, Hiepe F, Schlumberger W, Riemekasten G. Antibodies against PM/Scl-75 and PM/Scl-100 are independent markers for different subsets of systemic sclerosis patients. Arthritis Res Ther. 2009; 11(1):R22.

(12) Rozman B, Cucnik S, Sodin-Semrl S, Czirják L, Varjú C, Distler O, Huscher D, Aringer M, Steiner G, Matucci-Cerinic M, Guiducci S, Stamenkovic B, Stankovic A, Kveder T. Prevalence and clinical associations of anti-Ku antibodies in patients with systemic sclerosis: a European EUSTAR-initiated multi-centre case-control study. Ann Rheum Dis. 2008 September; 67(9):1282-6.

(13) Ihn H, Yamane K, Yazawa N, Kubo M, Fujimoto M, Sato S, Kikuchi K, Tamaki K. Distribution and antigen specificity of anti-U1RNP antibodies in patients with systemic sclerosis. Clin Exp lmmunol. 1999 August; 117(2):383-7.

(14) Fertig N, Domsic R T, Rodriguez-Reyna T, Kuwana M, Lucas M, Medsger T A Jr, Feghali-Bostwick C A. Anti-U11/U12 RNP antibodies in systemic sclerosis: a new serologic marker associated with pulmonary fibrosis. Arthritis Rheum. 2009 Jul. 15; 61(7):958-65.

1. An in vitro method for the diagnosis of Systemic Sclerosis (SSc) and/or Systemic Lupus Erythematosus (SLE) in a subject comprising:
reacting a test biological fluid sample taken from said subject with a mammalian cell nuclear extract reagent previously immobilized onto a solid support, under conditions allowing an antigen-antibody binding;
reacting a reference sample with a further mammalian cell extract reagent previously immobilized onto a solid support, under condition allowing an antigen-antibody binding;
washing;
adding detecting means able to detect the antigen-antibody binding, if occurred, and generating detecting values;
comparing detecting values of the test biological fluid sample with detecting values of the reference serum sample to obtain a mean fold reactivity value; and
checking if the mean fold reactivity value is higher or lower than a predefined cut-off value.

2. The method according to claim 1 wherein the test biological fluid sample is blood, plasma serum, optionally stored, frozen or lyophilized.

3. The method according to claim 1 wherein the mammalian cell nuclear extract reagent is obtained by cells comprised in the group of: cell line CHO, cell line HeLa, cell line 293 and cell line MRC-5.

4. The method according to claim 1 wherein the cell nuclear extract reagent is obtained by:
a) harvesting mammalian cells,
b) suspending harvested mammalian cells in a solution able to lyse cells maintaining intact nuclei, to get a mammalian cell lysate with intact nuclei;
c) pelleting the mammalian cell lysate and washing pelleted intact nuclei,
d) lysing nuclei in a proper solution to get a mammalian nuclear lysate, and
e) sonicating and centrifuging the nuclear lysate, discarding insoluble matter.

5. The method according to claim 4 wherein the solution in b) is a buffered, low salt hypotonic solution, completed with a protease inhibitors mix.

6. The method according to claim 4 wherein the solution in d) is a buffered, high salt hypertonic solution, completed with a protease inhibitors mix.

7. The method according to claim 1 wherein detecting means are peroxidase conjugated secondary antibodies.

8. The method according to claim 7 wherein the peroxidase conjugated secondary antibodies are peroxidase-conjugated anti-human IgGs.

9. The method according to claim 1 wherein the cut-off value is approximately 1.35 fold with respect to reference.

10. The method according to claim 1 for identifying the presence of vascular ulceration in SSc and Raynaud's phenomenon in SLE.

11. The method according to claim 1 wherein the SSc belongs to the group of: early SSc, limited cutaneous SSc, diffuse SSc, or CREST.

12. A kit for the diagnosis of Systemic Sclerosis (SSc) and/or Systemic Lupus Erythematosus (SLE) according to the method of claim 1.
